(19) United States
(12) Patent Application Publication
(10) Pub. No.: US 2026/0294342 A1
(43) Pub. Date: Oct. 1, 2026

(54) WEARABLE BIOSENSOR

(71) Applicant: TEKNOLOGIAN TUTKIMUSKESKUS VTT OY, Espoo (FI)

(72) Inventors: Timo KURKELA, Espoo (FI); Mohammadhossein BEHFAR, Espoo (FI)

(21) Appl. No.: 19/477,740

(22) PCT Filed: Apr. 17, 2024
(86) PCT No.: PCT/FI2024/050178

§ 371 Date: Oct. 22, 2025

(30) Foreign Application Priority Data
Apr. 24, 2023 (FI) 20235450

(51) Int. Cl. A61B 5/00

(52) U.S. Cl. CPC A61B 5/6833

(57) ABSTRACT

A skin patch structure including an elongated section and at least two wings with at least two conductors extending from the elongated section to the at least one wing. The disclosure further relates to a portable measurement device including a first lid and an electronics board each including at least two magnets, and a second lid configured to attach to the first lid. The advantage of the patch structure and the measurement device is that they can be electrically connected by enclosing the wings with conductors between the magnets of the first lid and the electronics board without any additional connecting elements in the patch design.

### FIELD OF THE DISCLOSURE

The present disclosure relates to a skin patch structure and a measurement device for detecting biological signals from the skin.

### BACKGROUND OF THE DISCLOSURE

Hybrid wearable devices often comprise a reusable electronics module and a disposable skin patch with electrodes, which adheres to the patient's skin. The mechanical and electrical connection between the electronics and the patch is often implemented through coupling mechanism which requires permanent integration of coupling components into patch design. Alternative ways of connection have been explored to simplify the patch structure and reduce the production costs by including less materials into the design. In US2014213878A1 latching was implemented through magnetic attraction of a first magnet in a disposable electrode patch and a second magnet in an electrode holder with the electrical trace. Such design requires integration of the connecting element, such as magnet, into the patch which increases the costs of production and complicates the disposal due to presence of extra parts.

### BRIEF DESCRIPTION OF THE DISCLOSURE

An object of the present disclosure is to provide a skin patch structure and a measurement device for implementing the biosignal measurement so as to solve the above problems.

The object of the disclosure is achieved by what is stated in the independent claims. The preferred embodiments of the disclosure are disclosed in the dependent claims.

The disclosure is based on the idea of using the patch structure which comprises the wings. The wings may be configured to connect to the measurement device, and specifically (a) to fold at the lid with one set of magnets, and (b) to connect to the electronics board with the another set of magnets.

An advantage of the arrangement of the disclosure is that the skin patch can be connected to the measurement device without additional connectors or connecting elements in the patch.

### BRIEF DESCRIPTION OF THE DRAWINGS

In the following the disclosure will be described in greater detail by means of preferred embodiments with reference to the accompanying drawings, in which

FIG. 1a illustrates the main elements of a skin patch structure such as an elongated section, the wings and the conductors;

FIG. 1b is a closer view of the wings and a folding portion with directions and side regions;

FIGS. 2a-2c illustrate different embodiments of the patch structure with two superimposed layers;

FIGS. 3a-3b illustrate another embodiment of the patch structure with three superimposed layers;

FIG. 4a is a general overview of the main elements of a measurement device such as a first lid, a second lid and an electronics board;

FIG. 4b illustrates an embodiment of the measurement device with additional elements;

FIGS. 5a-5b demonstrate the patch structure partly connected to the measurement device by folding the wings at the first lid at the folding portion.

### DETAILED DESCRIPTION OF THE DISCLOSURE

In this disclosure, single features of different subsections and embodiments may be combined to provide other embodiments. The embodiments may also contain features/structures that have not been specifically mentioned. All combinations of the embodiments are considered possible if their combination does not lead to structural or logical contradiction.

### Patch Structure

The skin patch structure of this disclosure comprises at least two superimposed layers, wherein a second layer is on a first layer; an elongated section, wherein the elongated section comprises at least two superimposed layers, and the first layer in the elongated section is configured to adhere to the skin; at least two wings extending sideways from the elongated section so that a first wing and a second wing are on the opposite sides of the elongated section, and wherein a folding portion is between the wings on the elongated section, and wherein the wings are configured to fold at the folding portion; at least two electrically separated conductors, wherein each conductor extends from the elongated section to one of the at least two wings, and the conductors are in the second layer.

### Two Superimposed Layers

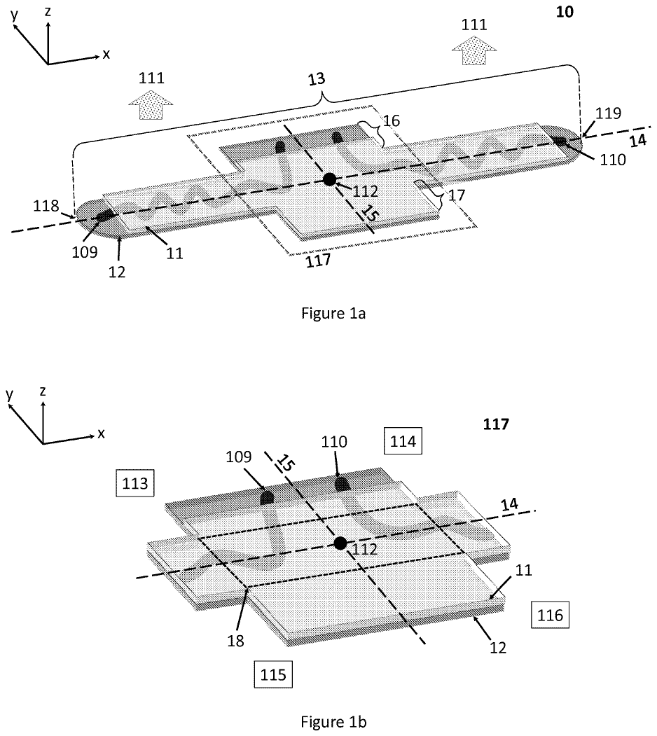

The patch structure of this disclosure, illustrated in FIGS. 1a-b, comprises at least two superimposed layers: a first layer 11 and a second layer 12. The first layer 11 is placed over the second layer 12. The first layer 11 is configured to adhere to the skin in the direction 111 and form a direct contact with the skin for biosignal measurement. The first layer 11 may comprise openings or apertures (not illustrated in FIG. 1). The first layer 11 may partly cover the second layer 12. The first layer 11 may have a top surface and a bottom surface. The second layer may also have a top surface and a bottom surface. The bottom surface of the first layer and the top surface of the second layer may be attached to each other in some sections of the patch structure.

The patch structure 10 may further comprise more than two layers (not illustrated in FIGS. 1a-b). One layer may be attached to the bottom surface of the second layer 12. Further, another layer may be applied to the top surface of the first layer 11 and configured as an adhesive between the patch 10 and the skin. Alternatively, the top surface of the first layer 11 may be inherently adhesive in order to adhere to the skin.

### Elongated Section

The patch structure 10 of this disclosure further comprises an elongated section 13. The elongated section 13 may be relatively thin and relatively long and may have a length significantly greater than a thickness. The elongated section 13 comprises at least two superimposed layers 11 and 12. In other words, the first layer 11 and the second layer 12 may form the elongated section. The first layer 11 in the elongated section is configured to adhere to the skin. The first layer 11 in the elongated section 13 may be shorter than the second layer 12. In that case, the second layer 12 in the elongated section may also be partly in contact with the skin.

The elongated section 13 defines a longitudinal direction 14, which extends along the length of the elongated section from one end 118 to another end 119. It also defines a lateral direction 15, which is orthogonal to the longitudinal direction. The longitudinal direction may also be called x-axis or x-direction, the lateral direction may also be called y-axis or y-direction, and z-axis may be orthogonal to both x- and −y axis as in FIG. 1a. The elongated section 13 may also define a horizontal plane (14, 15) or (x, y), in which all parts of the patch structure are in non-folded state as illustrated in FIG. 1.

The elongated section 13 may have a convex or a concave shape. A visual central point 112 of the shape may be defined as a point that is furthest from the edges of the elongated section in both lateral 15 and longitudinal 14 directions. In other words, the elongated section may have a rectangular shape, and the visual central point 112 may the match its geometric centre. However, the elongated section may also have an irregular shape, such as, for example, a “banana” shape, and its visual central point 112 may be inside the shape while the geometric centre may be outside the shape.

Four side regions 113-116 (FIG. 1b) may be formed by the nominal lines which pass through the visual central point 112 in the longitudinal 14 and lateral 15 directions. A first region 113 and a second region 114 may be on one side of the elongated section 13, and a third region 115 and a fourth region 116 may be on the other side of the elongated section 13.

The length of the elongated section 13 in the longitudinal direction 14 may be greater than its width in the lateral direction 15. The length and the width of the elongated section 13 may be different in different layers of the patch structure. In other words, the length and/or the width of the elongated section in the first layer 11 may be greater than those in the second layer 12. The length of the elongated section 13 may be 50-300 mm. The length of the elongated section 13 may be more than 20 mm, more than 30 mm, more than 50 mm, more than 70 mm, more than 100 mm, more than 150 mm, more than 180 mm, more than 200 mm. The length of the elongated section 13 may be less than 40 mm, less than 80 mm, less than 160 mm, less than 250 mm, less than 300 mm. The width of the elongated section may be 10-30 mm. The width of the elongated section may be more than 5 mm, more than 10 mm, more than 20 mm, more than 30 mm. The width of the elongated section may be less than 8 mm, less than 15 mm, less than 25 mm, less than 30 mm.

### The Wings

The patch structure of this disclosure comprises at least two wings 16 and 17. The patch structure may comprise 3 wings, 4 wings, or more than 4 wings. The at least two wings 16 and 17 extend sideways from the elongated section 13 so that at least a first wing 16 and a second wing 17 are on the opposite sides of the elongated section 13. In other words, one side of each wing may be connected to one side of the elongated section 13. The at least two wings 16 and 17 may extend in the direction perpendicular to the longitudinal direction.

The wings 16 and 17 may be along the horizontal plane (x, y) in non-folded state. The wings 16 and 17 may be close to the visual central point 112 of the elongated section, or close to the ends of the elongated section 13. The wings may also extend from the opposite sides of the elongated section 13 into at least one side region 113-116 on each side of the elongated section. The wings 16 and 17 may be orthogonal to the elongated section 13 and the longitudinal direction 14. The wings may alternatively be oriented at an angle of 30-90 degrees in the horizontal plane (x, y) with respect to the longitudinal direction 14.

The wings may comprise the second layer 12 of the patch structure only. Alternatively, at least one wing may comprise the second layer 12, and at least one other wing may comprise the other layer. Alternatively, the wings may comprise all layers of the patch structure. The wings may further comprise the entire second layer 12 and partially the first layer 11 and in FIGS. 1a-b. The at least two wings 16-17 and the elongated section 13 in the at least one of the at least two layers 11-12 may be fabricated as a continuous uniform structure. In other words, the wings may be configured non-separatable from the elongated section 13 in at least the second layer 12.

The wings may have substantially rectangular shape. It may further have rounded corners. The wings may also have semi oval shape or curved semi oval shape.

A length of the wings may be defined along the longitudinal direction 14 and a width of the wings may be defined along the lateral direction 15. The length and/or the width of the wings may be different in different layers of the patch structure. In other words, the length and/or the width of the wings 16-17 in the first layer 11 may be different than those in the second layer 12.

The length of each wing in x-direction may be 20-50 mm. The length of each wing in x-direction may be more than 20 mm, more than 25 mm, more than 30 mm, more than 35 mm, more than 40 mm, more than 45 mm. The length of each wing in x-direction may be less than 50 mm, less than 43 mm, less than 38 mm, less than 33 mm, less than 28 mm, less than 23 mm.

The width of each wing in y-direction may be 10-50 mm. Specifically, the width of each wing in y-direction may be more than 10 mm, more than 15 mm, more than 25 mm, more than 35 mm, more than 45 mm. The width of each wing in y-direction may be less than 50 mm, less than 40 mm, less than 30 mm, less than 20 mm, less than 12 mm.

The at least two wings may be configured to detachably mechanically connect to a measurement device separate from the patch structure. The wings may further be configured to detachably electrically connect to the measurement device separate from the patch structure. Importantly, the patch structure and the wings may comprise no connecting elements or connector configured to mechanically or electrically connect to the measurement device separate from the patch structure.

### Folding Portion and Folding of the Wings

A folding portion 18 is a portion of the elongated section 13 between the at least two wings 16 and 17. Specifically, the folding portion 18 may extend longitudinally between the at least two wings 16 and 17.

The at least two wings 16-17 are configured to fold at the folding portion 18. The wings may also be configured to fold at any additional element or device placed at the folding portion 18. Specifically, the at least two wings 16-17 may be configured to bend out of the horizontal plane (x, y) for about 180 degrees in both directions. In other words, the non-folded and folded state of at least two wings 16-17 may be at z=0 and during folding the wings may transition into a temporary folding plane (x, z). The at least two wings 16-17 may be further configured to wrap around any additional element or device placed at the folding portion 18. The wings bent out of the horizontal plane and folded at the folding portion may define the folded state.

### Conductors

The patch structure further comprises at least two electrically separated conductors 109 and 110. Each conductor extends from the elongated section 13 to one of the at least two wings 16-17. The at least two conductors 109-110 may extend to the same wing (for example 16). The conductors may also extend to different wings. The at least two conductors 109-110 may extend from the elongated section 13 to at least of the wings 16-17 across the folding portion 18. In other words, the conductors may cross the folding portion 18.

The patch structure may comprise more than 2 conductors, more than 4 conductors, more than 8 conductors, more than 10 conductors, more than 20 conductors. Each conductor may be electrically separated from the other conductors.

The at least two conductors 109-110 are in the second layer 12 of the patch structure 10. Specifically, the at least two conductors 109-110 may be on the top surface of the second layer 12. Since the top surface of the second layer 12 and the bottom surface of the first layer 11 are connected, the at least two conductors 109-110 may be partially connected to the bottom surface of the first layer 11. Additionally, part of the at least two conductors 109-110 in the elongated section 13 may further be covered with the volume of material or combination of materials other than the first layer (not shown in FIGS. 1a-b). Specifically, the material may be in some areas at the top surface of the second layer 12. One example of such material may be hydrogel. The hydrogel may be conductive and transfer the electrical signals from the skin to the conductor.

Each conductor may be configured to measure biosignals from the skin. Specifically, the conductors may be configured to measure biopotentials. Each conductor may be connected to the skin and to the measurement device separate from the patch structure.

The at least two wings 16-17 are configured to fold at the folding portion 18. Thus, in the folded state, the at least two conductors 109-110 may face away from the folding portion 18.

### Embodiments of the Patch

The skin patch structure of this disclosure, wherein the first and the second layers may be in the elongated section, and the second layer may be in the wings.

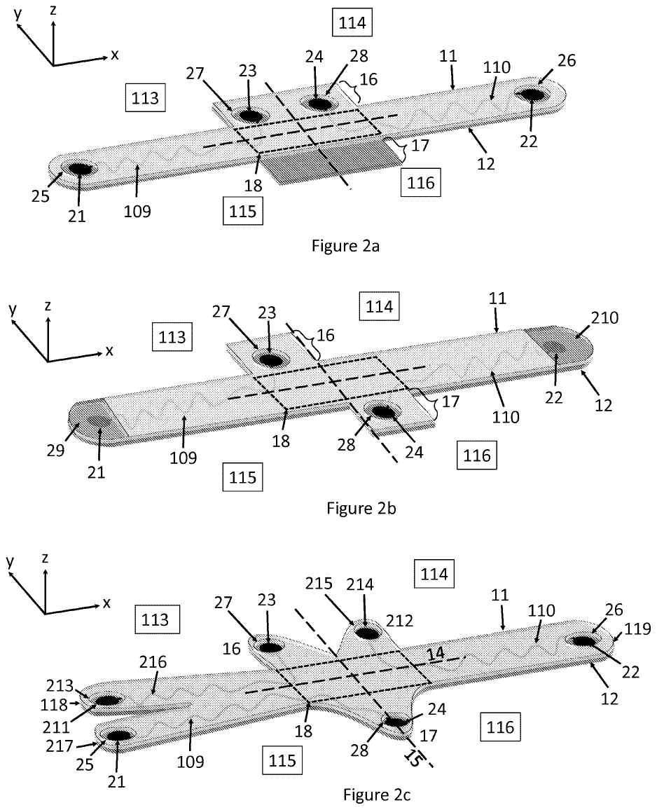

In any embodiments of this disclosure, the at least the first 11 and the second 12 layers may be in the elongated section 13, and at least the second layer 12 may be in the wings 16-17. Specifically, the patch structure 10 may comprise only the first layer 11 and the second 12 layer in the elongated section 13, and only the second layer 12 in the wings 16-17 as illustrated in FIGS. 2a-c.

### Embodiment 1

Each of the conductors of the patch structure of this disclosure may further comprise a sensing region in the elongated section and a contact region in one of the wings.

The first layer of the patch structure of this disclosure may further comprise an opening above each of the sensing regions.

As illustrated in FIG. 2a, each conductor of the patch structure may comprise a sensing region 21-22 in the elongated section and a contact region 23-24 in one of the at least two wings 16-17. The sensing regions 21-22 may be in contact with the skin, and the contact regions 23-24 may be in contact with the measurement device separate from the patch structure. Specifically, the contact regions 23-24 may be flat. In other words, the contact regions 23-24 may have same thickness as the other regions of the conductors 105-110.

The contact regions 23-24 may be symmetrical with respect to y-axis and equidistant from the elongated section 18 when located in one wing as in FIG. 2a. Alternatively, the contact regions 23-24 may be in the nominal centre of the wings when located in different wings as in FIG. 2b. Alternatively, the contact regions 23-24,214 may be in the nominal end of the wings furthest from the elongated section when located in different wings as in FIG. 2c.

In the folded state, the conductors 109-110 in the elongated section and the contact regions 23-24 in the wing 16 may be at the folding portion 18.

Further, the first layer may comprise an at least two openings 25-26 above each of the sensing regions. The first layer 11 may also comprise the openings 27-28 above each of the contact regions.

In the embodiment of FIG. 2a, the patch structure may comprise at least two wings 16-17, each extending into both regions 113-116 at each side of the elongated section. Each of the at least two conductors 109-110 may extend from elongated section 13 to one of the wings 16-17.

In any embodiment, the first layer 11 may be in at least partially in the elongated section 13 only. Alternatively, the first layer 11 may be at least partially in the elongated section 13 and at least partially in at least one of the wings (for example 16 in FIG. 2 a). Further, the first layer 11 may be at least partially in the elongated section 13 and at least partially in all of the at least two wings 16-17.

### Embodiment 2

As illustrated in FIG. 2b, the patch structure 10 of another embodiment may comprise the at least one wing 16-17 at each side of the elongated section, each extending into one of the four side regions 113-116. Specifically, the first wing 16 may extend into the first region 113, and the second wing 17 may extend into the fourth region 116. The first conductor 109 may extend from the elongated portion 13 to the first wing 16, and the second conductor 110 may extend from the elongated portion 13 to the second wing 110. Additionally, the patch structure 10 may comprise more wings extending into other two of the four regions (for example into regions 114-115).

As shown in FIG. 2b, the first layer 11 in the elongated section 13 may be shorter than the second layer 12. The at least two sensing regions 21-22 may each be covered with the volume of material 29-210 other than the material of the first layer 11.

### Embodiment 3

The patch structure of the embodiment of FIG. 2c may comprise more than two wings. Specifically, it may comprise three wings 16, 17 and 212. The two wings 16 and 212 may be on one side of the elongated section 13, and one wing 17 may be on the opposite side of the elongated section 13. The two wings 16 and 212 may be in two different side regions on one side of the elongated section 13, and one wing 17 may be in both side regions on the opposite side of the elongated section 13. Alternatively, one wing 17 may be in one side region on the opposite side of the elongated section 13. The wings 16, 17 and 212 may be oriented at an angle of 30-90 degrees in the horizontal plane (14, 15) with respect to the elongated section 13. Some of the three wings 16, 17 and 212 may each comprise the contact region (23, 214, 24). Alternatively, all of the three wings 16, 17 and 212 may each comprise the contact region (23, 214, 24). The first layer may partly cover the at least three wings 16, 17 and 212.

Further, the elongated section may have more than two ends. Specifically, the elongated section may have at least three ends 119, 118, 217. In other words, one of the physical ends of the elongated section 13 may be split into at least two sections (for example 118 and 217 in FIG. 2c). The number of conductors may correspond to the number of the wings. For example, each of the at least three conductors 109, 110 and 216 may extend to each of the at least three wings 16, 17 and 212.

### Fixing Apertures in the Patch

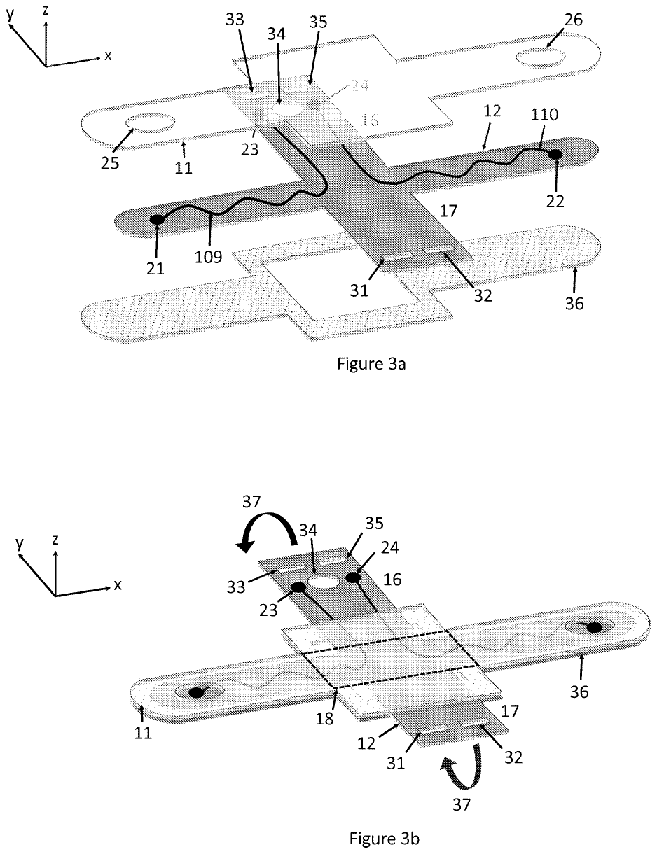

The patch structure may further comprise an at least one fixing aperture (such as 31 and 35) in each of the wings as illustrated in FIGS. 3a-b. The fixing apertures 31-35 may be in the second layer 12. The fixing aperture may also be in the first layer 11. The fixing aperture may also be in a third layer 36. Each of the wings may comprise two apertures (such as 31-32 and 33-34). Each of the wings may comprise more than two apertures. One wing may comprise more apertures than the other wing. The fixing apertures may, for example, be positioned as in FIGS. 3a-b. The apertures 33 and 35 in the first wing 16 may be aligned with the corresponding contact regions 23-24 along y-axis. Additionally, the apertures 33 and 35 may be further away from the elongated section 13 than the contact regions 23-24 are along y-axis. Alternatively, the apertures 31, 32, 33, 35 may be at the opposite sides of the wings 16-17equidistant from the elongated section 13. Another aperture, such as fixing aperture 34, may be in the nominal centre of the wings 16-17. Specifically, the aperture, such as fixing aperture 34, may be between the contact regions 23-24. The fixing apertures may be configured to mechanically fix the wings to the measurement device separate from the patch structure.

### Embodiment 4

The skin patch structure of this disclosure, may further comprise a third layer, wherein the second layer may be between the first layer and the third layer

The skin patch structure of this disclosure, wherein at least one fixing aperture may be in each of the wings.

As illustrated in another embodiment of FIGS. 3a-b, the patch structure may further comprise the third layer 36. The second layer 12 may be between the first 11 and the third 36 layers. In other words, the second layer 12 with the conductors 109-110 may be laminated between the first layer 11 and the third layer 36 as shown in FIG. 3a. The first 11 and the third 36 layer may at least partially embed in the second layer 12. The first layer 11 may have openings 25-26 above the sensing regions and the contact regions. The first layer 11 may be at least partly in all the at least two wings 16-17. The first layer 11 may also be in the elongated section 13 only or in one of the at least two wings 16-17.

The folding of the wings 16-17 may be out of the horizontal plane (x, y) as in the direction which arrow 37 indicate in FIG. 3b. In other words, the wings 16-17 may be configured to fold around the third layer 36 at the folding portion 18.

The patch structure may further comprise at least one fixing aperture in each of the at least two wings 16-17. As shown in FIGS. 3a-b, three apertures (33-35) may be in the first wing 16 and two apertures (31 and 32) in the second wing 17. The apertures may be in the second layer 12. The apertures may also be in the first layer 11 and the third layer 36.

### Materials and Properties of the Patch Structure

The patch structure may be flexible. Specifically, the elongated section and the wings may be capable of bending out of the horizontal plane and folding easily without breaking. The small thickness and choice of suitable materials of the patch structure enable the flexibility.

The patch structure may be disposable. All materials of the patch structure may be disposed under the same category of waste. Thus, the patch structure may be disposed without disassembly. This simplifies waste management and reduces costs.

The material of the at least the first and the second layers may be from bio-based group, such as corn or sugar cane-based biopolymers. The material of the at least the first and the second layers may be cellulose. The material of the at least the first and the second layers may be cellulose derivatives.

The first layer 11 and the second layer 12 may be attached to each other by adhesive. The adhesive may be bio-based. The adhesive may be hydroxypropyl cellulose (HPC) or nanocellulose. The first layer and the second layer may be attached to each other by bonding. The bonding agent may be acrylic-based or silicon-based. Alternatively, the bonding may be performed by heating the first and the second layer and pressing them together when heated. In other words, the bonding may be performed using heat lamination process.

The material of the conductors may be metal such as silver, copper, aluminium.

Alternatively, the skin patch structure of this disclosure may be metal-free. In other words, the first layer, the second layer and the conductors of the patch structure may not comprise any metallic elements.

The material of the conductors may be metal-free. The material of the conductors may be bio-based. The material of the conductors may further be electrically conductive. The material of the conductors may be carbon. Carbon may be derived from trees and animal sources, as well as from fossil-based sources. The material of the conductors may be graphene.

The material of the first layer may be non-conductive. The material of the first layer may further be adhesive. The non-conductive and adhesive properties of the first layers enable partial insulation of the conductors in the second layer and good attachment to the skin for biosignal measurement.

The material of the first layer and the second layer may be polymeric. Alternatively, the patch structure may be polymer-free. In other words, the first layer, the second layer and the conductors of the patch structure may not comprise any polymeric elements.

### Measurement Device

This disclosure further describes a portable miniaturized measurement device for measuring biosignals. The measurement device comprises an insulated first lid with a top side and a bottom side, wherein the first lid comprises at least two bottom magnets on the top side of the first lid; a measurement electronics board with a top side and a bottom side, wherein the measurement electronics board comprises at least two top magnets on the bottom side, and wherein the measurement electronics board comprises an electronics circuit, and electronics circuit is connected the top magnets, and each of the top magnets is configured to align with one of the bottom magnets; an insulated second lid, wherein the second lid is configured to detachably attach to the first lid, and the second lid and the first lid are configured to form an enclosure around the measurement electronics board.

### First Lid

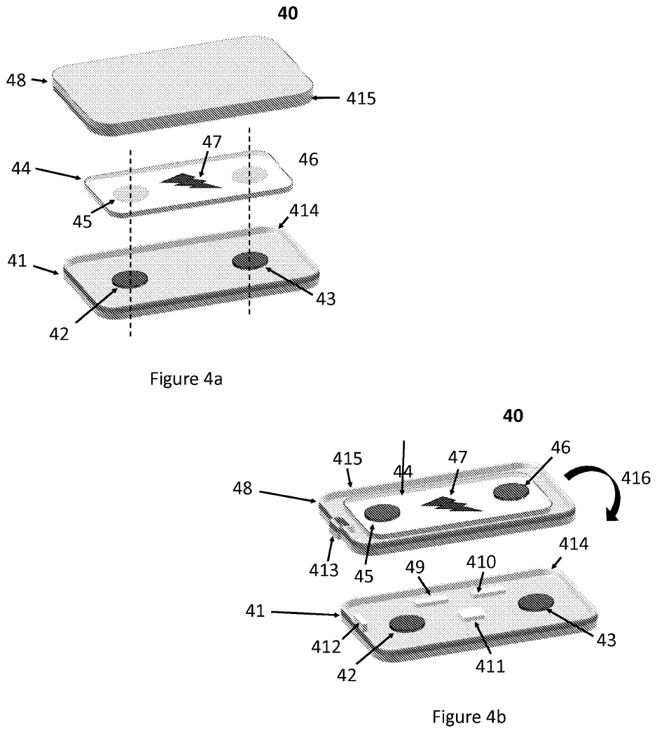

As illustrated in FIG. 4a, a portable measurement device 40 of this disclosure comprises a first lid 41. The first lid comprises an outer side and an inner side (visible in FIGS. 4a-b). It further comprises at least two bottom magnets 42 and 43 on the inner side. The first lid 41 may also comprise more than two magnets.

The first lid 41 may be configured to detachably mechanically connect to the patch structure 10 separate from the measurement device 40. Each of the at least two magnets 42 and 43 may be configured to align with one of the contact regions of the patch structure 10 separate from the measurement device.

### Electronics Board

The portable measurement device 40 for measuring biological signals further comprises a measurement electronics board 44 with a top side (visible in FIGS. 4a-b) and a bottom side. The measurement electronics board 44 comprises at least two top magnets 45-46 on the bottom side of the electronics board 44. The measurement electronics board may comprise three magnets on the bottom side. The measurement electronics board 44 may comprise more than three magnets on the bottom side. Each of the top magnets 45-46 is configured to align with one of the bottom magnets 42-43 in the first lid 41.

The measurement electronics board 44 comprises an electronics circuit 47, which is mechanically and electrically connected to the top magnets 45-46. The electronics circuit 47 may be on the top side of the measurement electronics board 44. The electronics circuit 47 may be on the bottom side of the measurement electronics board 44.

A width of the electronics board 44 may be 5-30 mm. The width of the electronics board 44 may be more than 5 mm, more than 10 mm, more than 20 mm, more than 25 mm. The width of the electronics board 44 may be less than 30 mm, less than 22 mm, less than 15 mm, less than 8 mm. A length of the electronics board 44 may be 10-40 mm. The length of the electronics board 44 may be more than 10 mm, more than 20 mm, more than 30 mm. The length of the electronics board 44 may be less than 40 mm, less than 35 mm, less than 25 mm, less than 15 mm.

A thickness of the electronics board 44 may be significantly less than its width and length. The thickness of the electronics board 44 may be 0.1-3 mm. The thickness of the electronics board 44 may be more than 0.1 mm, more than 0.3 mm, more than 0.8 mm, more than 1.2 mm, more than 1.6 mm, more than 2.5 mm. The thickness of the electronics board 44 may be less than 0.5 mm, less than 1 mm, less than 2 mm, less than 3 mm.

### Magnets

The wings 16-17 of the skin patch 10 structure may be configured to fold at the first lid 41 so that each of the contact regions 23-24 in the patch is in direct contact with one of the bottom magnets 42-43. The electronics board 44 with the top magnets 45-46 may be placed on the folded wings 16-17. Each of the top magnets 45-46 may be configured to connect with one of the bottom magnets 42-43 and grip one of the contact regions 23-24 in between, so that the folded wings 16-17 are attached between the top 45-46 and the bottom magnets 42-43.

The top and the bottom magnets may be permanent magnets. Alternatively, the material of the top magnets or the bottom magnets may be magnetic. In other words, the top magnets may be permanent magnets, and material of the bottom magnets may be magnetic. Alternatively, the bottom magnets may be permanent magnets, and material of the top magnets may be magnetic.

The material of the permanent magnets may be “hard” ferromagnetic. “Hard” ferromagnetic materials are characterized by being constantly magnetized and creating their own magnetic field. Specifically, the material of the permanent magnets may be, for example, ceramic, neodymium, ferrite or samarium cobalt.

The magnetic materials may be “soft” ferromagnetic. “Soft” ferromagnetic materials may be easily magnetized and demagnetized when placed into and removed from the external magnetic field. The magnetic materials may be, for example, iron, nickel or steel.

Alternatively, the magnets may be formed by depositing a magnetic ink. The magnetic ink may be deposited to the electronics board 44 to form the top magnets 45-46. The magnetic ink may be deposited to the first lid 41 to form the bottom magnets 42-43. The magnetic ink may be deposited using, for example, printing. The magnetic ink may comprise magnetic iron oxide.

### Second Lid

The measurement device 40 further comprises a second lid 48. The second lid comprises an outer side (shown in FIG. 4a) and an inner side (shown in FIG. 4b). The second lid may be configured to detachably mechanically connect to the first lid and enclose the wings of the patch structure separate from the measurement device.

### Dimensions of the Lids

The first lid 41 and the second lid 48 may have a substantially rectangular shape. The rectangular shape may have rounded corners. Alternatively, the lids may have an oval shape.

A width of the first 41 and the second 48 lids may be 5-40 mm. The width of the first 41 and the second 48 lids may be more than 5 mm, more than 10 mm, more than 20 mm, more than 25 mm, more than 30 mm. The width of the first 41 and the second 48 lids may be less than 35 mm, less than 30 mm, less than 24 mm, less than 17 mm, less than 9 mm. A length of the first 41 and the second 48 lids may be 10-50 mm. The length of the first 41 and the second 48 lids may be more than 10 mm, more than 20 mm, more than 30 mm, more than 40 mm. The length of the first 41 and the second 48 lids may be less than 50 mm, less than 45, less than 35 mm, less than 25 mm, less than 15 mm.

A thickness of the lids may be significantly less than their width and length. The thickness of the first 41 and the second 48 lids may be 0.1-5 mm. The thickness of the first 41 and the second 48 lids may be more than 0.1 mm, more than 0.5 mm, more than 1.5 mm, more than 2.5 mm, more than 3.5 mm, more than 4.5 mm. The thickness of the first 41 and the second 48 lids may be less than 5 mm, less than 4 mm, less than 3 mm, less than 2 mm, less than 1 mm, less than 0.7 mm.

Each of the lids may further comprise at least one side wall 414-415 at the edge of their inner side (shown in FIGS. 4a-b). The side walls 414-415 may come into contact when the lids 41 and 48 are connected. In other words, an enclosure space may be formed when the side walls 414-415 of the first lid 41 and the second lid 48 are attached to each other. Further, the side walls may only be at two opposite edges of the inner side of at least the first lid 41 (not illustrated). The side walls may be along the entire edge of the second lid 48 and may be recessed at two opposite sides of the first lid 41 so that the first 41 and the second 48 lids form an enclosure with openings in two opposite sides (not illustrated). The openings in the opposite sides of the enclosure may be configured to accommodate the wings of the patch, separate from the measurement device, to enter the inside of the enclosure. The height of the walls may be 0.5-3 mm. The height of the walls may be more than 0.5 mm, more than 1 mm, more than 2 mm. The height of the walls may be less than 3 mm, less than 2.5 mm, less than 1.5 mm, less than 0.8 mm.

### Materials of the Lids

The first 41 and the second 48 lid materials are electrically insulating. They may also be thermally insulating.

The material of the lids may be plastics. Specifically, the material of the lids may be, for example, polyethylene terephthalate (PET). Alternatively, the material of the lids may be bio-based. The material of the lids may be sugar cane or corn based polylactic acid (PLA).

### Embodiment of the Measurement Device

The portable measurement device of claim 9, wherein the measurement electronics board may be inseparably mechanically fixed to the second lid.

As illustrated in FIG. 4b, the second lid 48 and the electronics board 44 of the measurement device 40 may be inseparably connected. In other words, the electronics board 44 may form part of the inner side of the second lid 48. The electronics circuit 47 and the at least two magnets 45-46 may be on bottom side of the electronics board 44. The second lid 48 with the electronics board 44 may be connected to the first lid 41 but rotating the second lid 48 upside down in the direction of the arrow 416 in order to enclose the electronics board 44 in between.

The portable measurement device of any preceding claim, wherein the first lid may further comprise at least two fixing pins.

As further illustrated in FIG. 4b, the first lid 41 may comprise at least two fixing pins on the inner side (at least two of 49-411). The fixing pins 49-411 may be configured to mechanically connect the wings 16-17 of the patch structure 10 separate from the measurement device 40. The fixing apertures 31-35 of the patch structure 10 may connect to the fixing pins 49-411 of the first lid 41. The first lid 41 may comprise more than two fixing pins.

### Locking of the Lids

Each of the lids may further comprise at least one locking element at their inner side as illustrated in FIG. 4b. The at least one locking element may be at the side wall of each the first lid 41 and the second lid 48. The locking elements may, for example, be a locking pin 412 in the first lid 41 and a locking aperture 413 in the second lid 48. The locking elements 412 and 413 may be configured to align with each other and connect. Specifically, the locking pin 412 may be pushed into the locking aperture 413. In other words, the locking elements may be configured to detachably fix the lids together.

The second lid 48 with the electronics board 44 may further be permanently connected to the first lid 41 at one connection point. The connection point may be, for example, a hinge element attached to the inner side of the first lid 41 and to the inner side of the second lid 48 (not illustrated in the Figures). The connection point may be configured to reversibly separate the lids. In other words, the lids may be configured to open while being connected at one point.

### Functionality of the Measurement Device

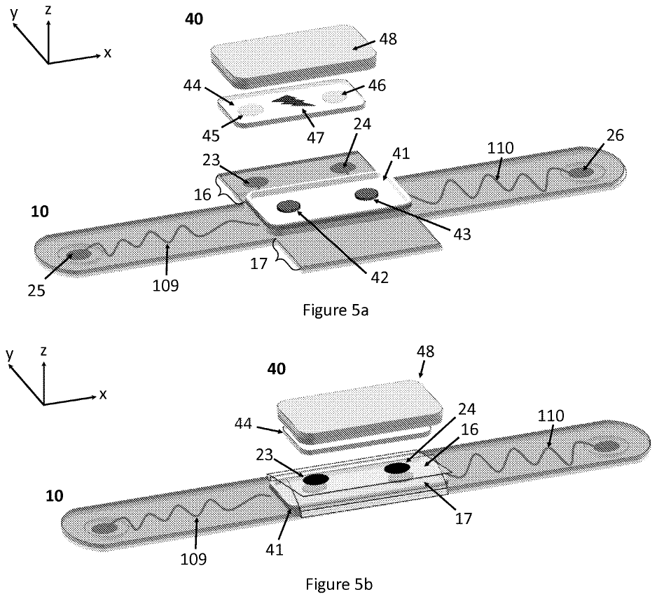

The measurement device may be configured to enclose at least the first wing 16 and the second wing 17 of the patch structure 10 (illustrated in FIGS. 5a-b) and to grip each of the at least two contact regions 23-24 between one of the bottom magnets 42-43 and one of the top magnets 45-46. The gripping provides mechanical and electrical coupling between the contact regions 23-24 of the patch structure 10, separate from the measurement device 40, and the electronics circuit 47 connected to the top magnets 45-46 for transmission of biopotential signals from the patch structure 10 to the measurement device 40.

The measurement device 40 is modular. The measurement device 40 is configured to reversibly assemble by attaching the first lid 41 to the second lid 48 with the electronics board 44 in between when connected to the patch structure 10. The measurement device 40 is also configured to reversibly disassemble when the patch structure 10 is disconnected. In other words, the measurement device may be configured to enclose the wings of one patch, release the wings by disconnecting the first lid, the electronics board and the second lid, and to enclose the wings of the other patch by connecting the first lid, the electronics board and the second lid again.

### Wearable Biosensor

A wearable biosensor, illustrated in FIGS. 5a-b, may comprise (a) a skin patch structure 10 with
at least two superimposed layers, wherein a second layer 12 is on a first layer 11;
an elongated section 13, wherein the elongated section 13 comprises at least two superimposed layers 11-12, and the first layer 11 in the elongated section 13 is configured to adhere to the skin;
at least two wings 16-17 extending sideways from the elongated section 13 so that a first wing 16 and a second wing 17 are on the opposite sides of the elongated section 13, and wherein a folding portion 18 is between the wings 16-17 on the elongated section 13, and wherein the wings 16-17 are configured to fold at the folding portion 18;
at least two electrically separated conductors 109-110, wherein each conductor extends from the elongated section 13 to one of the at least two wings 16-17, and the conductors are in the second layer 12;
   and (b) a portable miniaturized measurement device 40 with
an insulated first lid 41 with a top side and a bottom side, wherein the first lid comprises at least two bottom magnets 42-43 on the top side of the first lid;
a measurement electronics board 44 with a top side and a bottom side, wherein the measurement electronics board 44 comprises at least two top magnets 45-46 on the bottom side, and wherein the measurement electronics board comprises an electronics circuit 47, and electronics circuit 47 is connected the top magnets 45-46, and each of the top magnets 45-46 is configured to align with one of the bottom magnets 42-43;
an insulated second lid 48, wherein the second lid 48 is configured to detachably attach to the first lid 41, and the second lid 48 and the first lid 41 are configured to form an enclosure around the measurement electronics board 44
   for measuring biosignals;
   and wherein the wings 16-17 of the skin patch structure may be configured to extend into the enclosure formed by the first lid 41 and the second lid 48, and the wings 16-17 may further be configured to extend over the bottom magnets 42-43 so that the conductors 109-110 form electrical and mechanical contact with the top magnets 45-46 and the electronics circuit 47. Importantly, the wings 16-17 of the skin patch may be configured to directly connect to the magnets of the measurement device. Specifically, no additional connector or connecting elements in the skin patch are required.

In other words, the wearable sensor of this disclosure may be configured to combine the skin patch structure 10 and the measurement device 40 interconnected with each other as FIGS. 5a-b illustrates. The wings 16-17 of the patch structure 10 may be configured to fold at the first lid 41 placed at the folding portion 18. The conductors 109-110 in the wings 16-17 of the patch structure 10 may be configured to align with the bottom magnets 42-43 in the first lid 41. The conductors 109-110 in the wings 16-17 may also be configured to align and form both mechanical and electrical connection with the top magnets 45-46 of the electronics board 44. Since the top magnets 45-46 and the electronics circuit 47 are electrically connected, the conductors 109-110 may be configured to form electrical connection with the electronics board 44. Specifically, the contact regions 23-24 may be configured to align with the bottom magnets 42-43 and form electrical connection with the electronics circuit 47. Both the first lid 41 and the second lid 48 may be configured to enclose the wings 16-17 of the patch structure 10 and the electronics board 44 in between. Importantly, the wings of the patch may comprise no additional connecting elements configured to connect to the measurement device. Furthermore, the wings may be configured to connect to the measurement device with no separate connector.

### Method of Connecting the Skin Patch to the Measurement Device

A method of connecting the skin patch structure 10 and the measurement device 40, illustrated in FIGS. 5a-b, may comprise the following steps:
a) placing the outer side of the first lid 41 at the folding portion 18;
b) folding one of the wings 17 at the inner side of the first lid 41;
c) aligning each of the conductors 109-110 with one of the bottom magnets 42-43;
d) folding the other wing 16 at the inner side of the first lid 41;
e) aligning each of the conductors 109-110 with one of the bottom magnets 42-43 of the first lid 41;
f) attaching the electronics board 44 by aligning the bottom magnets 42-43 of the first lid 41 and the top magnets 45-46 of the electronics board 44 and gripping the conductors 109-110 in between.

Additionally, the method may include the following step:
g) attaching the second lid 48 and enclosing the wings 16-17 with conductors 109-110 in between the lids 41-48.

Specifically, the steps c), e), f) and g) of the method may relate to the contact regions 23-24 of the conductors 109-110.

The method may further include a step, in which each aperture in the wings (for example 33-35 in FIG. 3b) is detachably mechanically attached to each of the pins (for example 49-411 in FIG. 4b) on the top side of the first lid 41 (not illustrated in FIGS. 5a-b).

1-12. (canceled)

13. A skin patch structure for cooperation with a measurement device comprising:
at least two superimposed layers comprising a first layer and a second layer, wherein the second layer is on the first layer;
an elongated section, wherein the elongated section comprises at the least two superimposed layers, and the first layer in the elongated section is configured to adhere to the skin;
at least two wings comprising a first wing and a second wing extending sideways from the elongated section so that first wing and the second wing are disposed on opposite sides of the elongated section, and further comprising a folding portion between the at least two wings on the elongated section, and wherein the at least two wings are configured to fold at the folding portion;
at least two electrically separated conductors, wherein each conductor of the at least two electrically separated conductors extend from the elongated section to one of the at least two wings, and each of the conductors of the at least two electrically separated conductors are in the second layer,
wherein the at least two wings are configured to detachably mechanically and electrically connect to the measurement device separate from the patch structure.

14. The skin patch structure of claim 13, wherein each of the at least two electrically separated conductors comprise a sensing region in the elongated section and a contact region in one of the wings.

15. The skin patch structure of claim 14, wherein the first layer comprises an opening disposed proximate each of the sensing regions.

16. The skin patch structure of claim 13, wherein the first and the second layers are disposed in the elongated section, and the second layer is in the wings.

17. The skin patch structure of claim 13, further comprising a third layer, wherein the second layer is disposed between the first layer and the third layer.

18. The skin patch structure of claim 13, wherein at least one fixing aperture is disposed in each of the at least two wings.

19. The skin patch structure of claim 13, wherein the skin patch structure is metal-free.

20. The skin patch structure of claim 13, wherein a length of the at least two wings is 20-50 mm, and a width of the at least two wings is 10-50 mm.

21. A portable miniaturized measurement device for cooperation with a skin patch structure having wings with conductors for measuring biosignals comprising:
an insulated first lid having a top side and a bottom side, wherein the insulated first lid comprises at least two bottom magnets on the top side of the insulated first lid;
a measurement electronics board having a top side and a bottom side, wherein the measurement electronics board comprises at least two top magnets on the bottom side, and
wherein the measurement electronics board comprises an electronics circuit, and the electronics circuit is operatively connected the top magnets, and each of the at least two top magnets is configured to align with one of the at least two bottom magnets;
an insulated second lid, wherein the second lid is configured to detachably attach to the first lid, and the second lid and the first lid are configured to form an enclosure around the measurement electronics board;
wherein the conductors of the wings of the skin patch structure are configured to align with the at least two bottom magnets of the insulated first lid and the at least two top magnets of the measurement electronics board.

22. The portable measurement device of claim 21, wherein the measurement electronics board is inseparably mechanically fixed to the second lid.

23. The portable measurement device of claim 21, wherein the insulated first lid further comprises at least two fixing pins.

24. A wearable biosensor comprising a skin patch structure for cooperation with a measurement device, comprising:
the skin patch structure comprising:
at least two superimposed layers comprising a first layer and a second layer, wherein the second layer is on the first layer;
an elongated section, wherein the elongated section comprises at the least two superimposed layers, and the first layer in the elongated section is configured to adhere to the skin;
at least two wings comprising a first wing and a second wing extending sideways from the elongated section so that first wing and the second wing are disposed on opposite sides of the elongated section, and further comprising a folding portion between the at least two wings on the elongated section, and wherein the at least two wings are configured to fold at the folding portion;
at least two electrically separated conductors, wherein each conductor of the at least two electrically separated conductors extend from the elongated section to one of the at least two wings, and each of the conductors of the at least two electrically separated conductors are in the second layer,
the measurement device comprising:
an insulated first lid having a top side and a bottom side, wherein the insulated first lid comprises at least two bottom magnets on the top side of the insulated first lid;
a measurement electronics board having a top side and a bottom side, wherein the measurement electronics board comprises at least two top magnets on the bottom side, and
wherein the measurement electronics board comprises an electronics circuit, and the electronics circuit is operatively connected the top magnets, and each of the at least two top magnets is configured to align with one of the at least two bottom magnets;
an insulated second lid, wherein the second lid is configured to detachably attach to the first lid, and the second lid and the first lid are configured to form an enclosure around the measurement electronics board;
wherein the at least two electrically separated conductors of the at least two wings of the skin patch structure are configured to align with the at least two bottom magnets of the insulated first lid and the at least two top magnets of the measurement electronics board, and
wherein the at least two wings are configured to detachably mechanically and electrically connect to the measurement device separate from the patch structure
